(19) United States
(12) Patent Application Publication
(10) Pub. No.: US 2024/0013869 A1
(43) Pub. Date: Jan. 11, 2024

(54) Predicting Shelf Life Stability of Lyophilized Drug Products

(71) Applicant: AMGEN INC., Thousand Oaks, CA (US)

(72) Inventors: Ron Christopher Kelly, Westlake Village, CA (US); Mary Chaves, Arlington, MA (US); Marc A. Caporini, Belmont, MA (US); Fabrice Schlegel, Cambridge, MA (US)

(21) Appl. No.: 18/025,311

(22) PCT Filed: Aug. 30, 2021
(86) PCT No.: PCT/US2021/048181

§ 371 Date: Mar. 8, 2023

Related U.S. Application Data
(60) Provisional application No. 63/075,956, filed on Sep. 9, 2020.

(51) Int. Cl. G16C 60/00; G16C 20/70; G16C 20/80

(52) U.S. Cl. CPC G16C 60/00; G16C 20/70; G16C 20/80

(57) ABSTRACT

A method of computational modeling to predict stability of a lyophilized drug product includes receiving model parameters describing a virtual cake, a virtual vial, a virtual stopper, and a virtual ambient environment. The method also includes computing, by implementing a computational model and at each of a plurality of virtual time steps, a change in water amount or concentration in the virtual cake, virtual air within the virtual vial, and the virtual stopper, in part by applying the model parameters to the computational model. The method also includes generating information for display to a user via a user interface.

### FIELD OF THE DISCLOSURE

The present application relates generally to lyophilized drug products, and more specifically to stability over the shelf life of lyophilized drug products.

### BACKGROUND

During manufacture, certain pharmaceutical drug products are lyophilized, or “freeze dried,” in order to increase stability and shelf life. In a typical lyophilization process, a vial containing the drug product is placed within a special lyophilization chamber, and the drug product is frozen by reducing the temperature within the chamber. The chamber is then evacuated, after which heat is added to the drug product to cause water (ice) in the drug product to sublimate (i.e., transition directly from the solid state to a gaseous state). By removing moisture from the drug product in this manner, the drug product is generally made more stable. However, the resulting drug product “cake” retains an intrinsic amount of moisture after the lyophilization cycle, and is susceptible over time to additional moisture migrating from the typically rubber closure (stopper) and the external ambient environment. If the moisture content in the cake becomes too high at any point during the shelf life of the drug product, physical instability (collapse) and/or chemical instability of the cake/product can result. Unfortunately, measuring the moisture content of the cake over the shelf life can be expensive in terms of labor, equipment, etc., and is inherently time consuming given the relatively long shelf life expected of most drug products (typically on the order of two to five years). Thus, any attempt to prolong drug product shelf lives by the judicious selection or design of various components/processes/conditions (e.g., vial type, stopper type, starting cake moisture, storage conditions, etc.) typically requires a long and costly process of trial and error. Accordingly, there is a need for methods and systems that can quickly and accurately estimate shelf life stability for new drug products (and/or particular vial/stopper combinations, storage conditions, etc.).

### BRIEF SUMMARY

Systems and methods described herein generally provide in silico modeling of a vial, stopper, and lyophilized drug product (cake) to predict the water content of the cake over a particular time horizon (e.g., over a desired or expected shelf life for the drug product). The model solves for water concentration (or amount) in three domains of the virtual, modeled environment: cake, air (within the vial), and stopper. The model may be a finite-element analysis (FEA) model, for example, and a solver may solve for water concentrations in the cake, air, and stopper domains by discretizing the time and space domains into a resolved mesh, and then solving the resulting equations subject to the appropriate boundary and initial conditions. In some implementations, the equations output a water concentration profile and water mass flux as a function of time. The model may assume an axisymmetric configuration of both vial and stopper, in which case the equations can be solved in two dimensions and integrated over 180 degrees of rotation relative to the vial/stopper central axis.

Inputs to the model may include the geometries (physical configurations) of the vial/stopper combination, as well as various properties of the drug product, the stopper, and the storage conditions under consideration. For example, model inputs provided by a user and/or the model developer may include the starting fill weight and moisture content of the drug product/cake, sorption/desorption moisture isotherms for the stopper-air and cake-air interfaces, the moisture diffusivity coefficient of the stopper, the temperature and relative humidity of the ambient storage environment, and so on.

The model predictions can provide immediate insight into the shelf life stability of a drug product and can be used in various ways. For example, the model inputs may be varied over successive model runs in order to facilitate the design or selection of various components or process parameters (e.g., to design, select, or qualify a stopper and/or vial that reduces the rate of moisture uptake into the cake, or to set a stopper drying target, etc.), or to determine optimal or desired storage conditions for a product/vial/stopper combination (e.g., temperature and relative humidity), and so on, thereby significantly reducing the development cycle. As another example, the impact of process deviations during manufacturing (e.g., unexpected cake or stopper moisture levels) on shelf life stability can be determined almost instantly and in a non-destructive manner, thereby facilitating fast and informed program decisions that can ensure product quality and consistent supply to patients (e.g., by adjusting other process parameters as needed to reduce cake moisture prior to storage, or by rejecting a batch or sample that is not expected to retain adequate stability for the advertised shelf life, etc.).

### BRIEF DESCRIPTION OF THE DRAWINGS

The skilled artisan will understand that the figures described herein are included for purposes of illustration and are not limiting on the present disclosure. The drawings are not necessarily to scale, emphasis instead being placed upon illustrating the principles of the present disclosure. It is to be understood that, in some instances, various aspects of the described implementations may be shown exaggerated or enlarged to facilitate an understanding of the described implementations. In the drawings, like reference characters throughout the various drawings generally refer to functionally similar and/or structurally similar components.

FIG. 1 is a simplified block diagram of an example system that may be used to predict stability-related attributes of a lyophilized drug product in a stoppered vial over time.

FIG. 2 depicts an example vial containing a lyophilized drug product, which can be simulated using the system of FIG. 1.

FIG. 3A is an example of a stopper-specific moisture isotherm that may be used by the computational model of FIG. 1 to compute stopper-air boundary conditions.

FIG. 3B is an example of a drug product-specific moisture isotherm that may be used by the computational model of FIG. 1 to compute cake-air boundary conditions.

FIG. 4A depicts an example user interface that the system of FIG. 1 may present to a user in order to obtain model inputs.

FIGS. 4B and 4C depict example outputs that the system of FIG. 1 may present to a user in order to facilitate shelf life stability assessments, and/or to facilitate the design or selection of components and/or process parameters.

FIG. 5 is a flow diagram of an example method of predicting stability of a lyophilized drug product stored in a vial.

### DETAILED DESCRIPTION

The various concepts introduced above and discussed in greater detail below may be implemented in any of numerous ways, and the described concepts are not limited to any particular manner of implementation. Examples of implementations are provided for illustrative purposes.

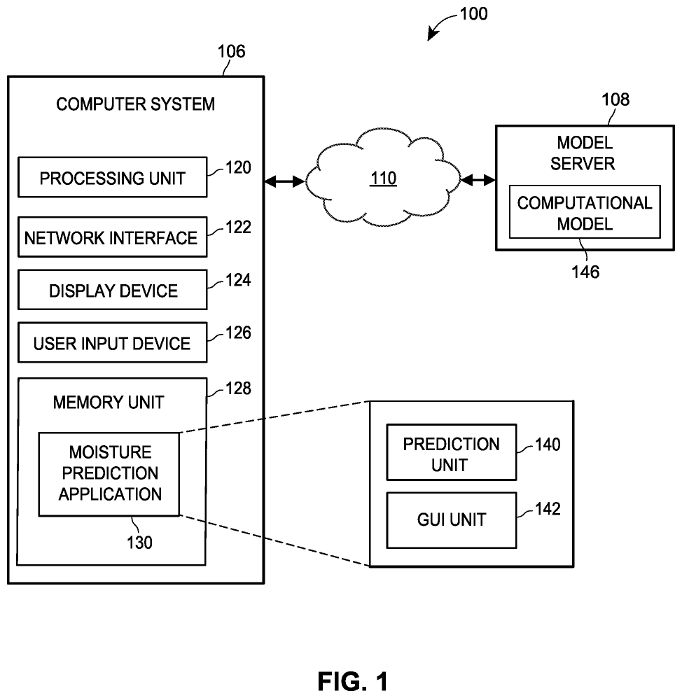

FIG. 1 is a simplified block diagram of an example system 100 that may be used to predict stability-related attributes of a lyophilized drug product in a stoppered vial over time. As used herein, the term “vial” may refer to any container suitable for holding a lyophilized drug product. The example system 100 includes a computing system 106 and a model server 108, coupled to each other via a network 110. The network 110 may be a single communication network or may include multiple communication networks of one or more types (e.g., one or more wired and/or wireless local area networks (LANs), and/or one or more wired and/or wireless wide area networks (WANs) such as the Internet or an intranet, for example).

The computing system 106 may be a server, a desktop computer, a laptop computer, a tablet device, or any other suitable type of computing device or devices. In the example embodiment shown in FIG. 1, the computing system 106 includes a processing unit 120, a network interface 122, a display device 124, a user input device 126, and a memory unit 128. In some embodiments, however, the computing system 106 includes two or more computers that are either co-located or remote from each other. In these distributed embodiments, the operations described herein relating to the processing unit 120, network interface 122, and/or memory unit 128 may be divided among multiple processing units, network interfaces, and/or memory units, respectively.

The processing unit 120 includes one or more processors, each of which may be a programmable microprocessor that executes software instructions stored in the memory unit 128 to execute some or all of the functions of the computing system 106 as described herein. Alternatively, some of the processors in the processing unit 120 may be other types of processors (e.g., application-specific integrated circuits (ASICs), field-programmable gate arrays (FPGAs), etc.), and some of the functionality of the computing system 106 as described herein may instead be implemented, in part or in whole, in such hardware. The memory unit 128 may include one or more physical memory devices or units containing volatile and/or non-volatile memory. Any suitable memory type or types may be used, such as read-only memory (ROM), solid-state drives (SSDs), hard disk drives (HDDs), and soon.

The network interface 122 may include any suitable hardware (e.g., front-end transmitter and receiver hardware), firmware, and/or software configured to communicate via the network 110 using one or more communication protocols. For example, the network interface 122 may be or include an Ethernet interface.

The display device 124 may use any suitable display technology (e.g., LED, OLED, LCD, etc.) to present information to a user, and the user input device 126 may be a keyboard or other suitable input device. In some embodiments, the display device 124 and the user input device 126 are integrated within a single device (e.g., a touchscreen display). Generally, the display device 124 and the user input device 126 may jointly enable a user to interact with graphical user interfaces (GUIs) provided by the computing system 106, e.g., in order to enter model inputs and view model outputs as discussed in further detail below.

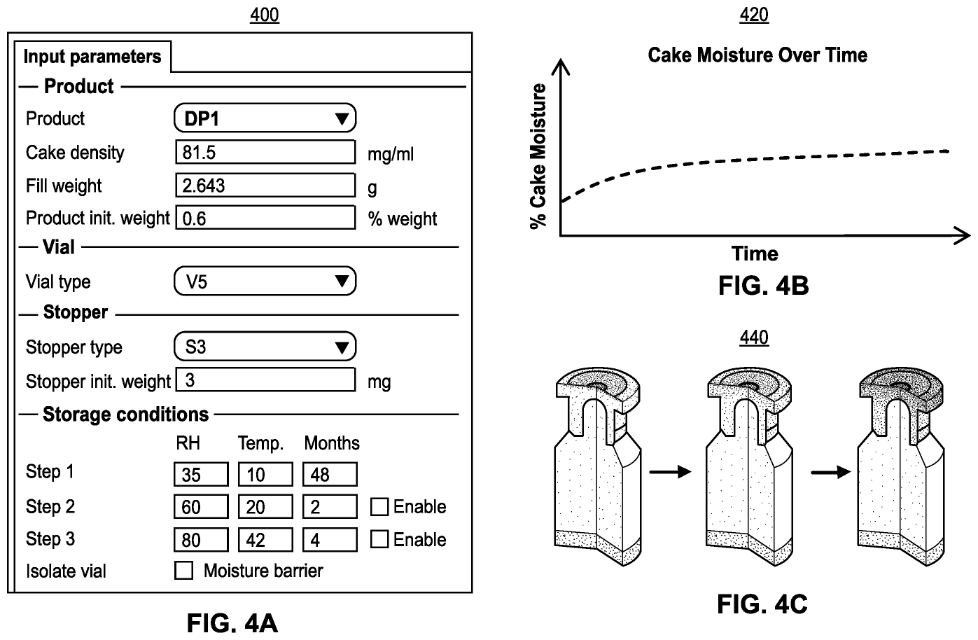

The memory unit 128 stores the instructions of one or more software applications, including a moisture prediction application 130, which in turn includes a prediction unit 140 and a GUI unit 142. The prediction unit 140 is generally configured to execute, or cause the execution of, a computational model 146 to predict one or more stability-related attributes of a lyophilized drug product, including a cake moisture content (i.e., water concentration or amount), for a given set of model inputs. The model inputs describe a virtual environment of interest, including a virtual vial that contains a virtual cake (i.e., lyophilized drug product) and virtual air, where the virtual vial is sealed by a virtual stopper. The GUI unit 142 is generally configured to generate one or more GUIs via which the moisture prediction application 130 can obtain user-entered information (e.g., entered via user input device 126), with the moisture prediction application 130 deriving or determining some or all of the model inputs from that user-entered information. In some implementations, some or all model inputs are provided in a different manner (e.g., via a file transferred from a remote source, etc.). The GUI unit 142 is also configured to generate one or more GUIs via which the moisture prediction application 130 presents model outputs for a user to view. Some example GUIs are shown in FIGS. 4A through 4C and discussed below. It is understood that the units 140, 142 of the moisture prediction application 130 may be implemented by different software applications, and/or that the functionality of any one such unit may be divided among two or more different software applications.

In the example implementation shown in FIG. 1, the computational model 146 resides at the model server 108. In some implementations, the model server 108 executes the computational model 146, and exchanges model input and output data with the computing system 106, as part of a web services model. In other implementations, the model server 108 transfers a copy of the computational model 146 to the computing system 106 via network 110, and the prediction unit 140 directly executes the computational model 146. In still other implementations, the system 100 does not include the model server 108, and the computational model 146 resides only at the computing system 106 (e.g., stored in the memory unit 128 or another suitable memory), and the prediction unit 140 directly executes the computational model 146.

The computational model 146 is a mechanistic/first-principles model that uses a diffusion equation and the model inputs to simulate, over time (i.e., over a number of virtual time steps), water content and water transmission within a virtual vial that is sealed with a virtual stopper and contains air and a virtual lyophilized drug product. As used herein, the term “air” may refer to any suitable gas. While the term “virtual” is not always used herein when referring to the simulated vial, stopper, and cake, it is understood that the simulations described herein are in silico, and thus that there need not be (but may be) a corresponding, real-world vial, stopper, or cake.

In some implementations, the computational model 146 solves for water concentration in three domains/media of the virtual environment: the cake within the vial, the air within the vial, and the stopper. The computational model 146 may solve for the water concentrations in these domains by discretizing time and space into a resolved mesh and solving the resulting equations subject to the appropriate initial and boundary conditions. The computational model 146 may output a water concentration profile and water mass flux in each domain as a function of time, for example. In some implementations, the prediction unit 140 (or model server 108) implements a solver that uses a finite-element analysis (FEA) discretization of a two-dimensional diffusion equation to perform computational fluid dynamics calculations over a series of virtual time steps. The computational model 146 may solve in two dimensions by assuming an axisymmetric configuration of the vial and stopper (and integrating over 180 degrees of rotation about the central axis) or, in other implementations, may account for asymmetries around the central axis of the vial/stopper (e.g., to account for an igloo-shaped formation within a stopper). Example equations that may be used in the computational model 146, including a diffusion equation and boundary conditions, are discussed below.

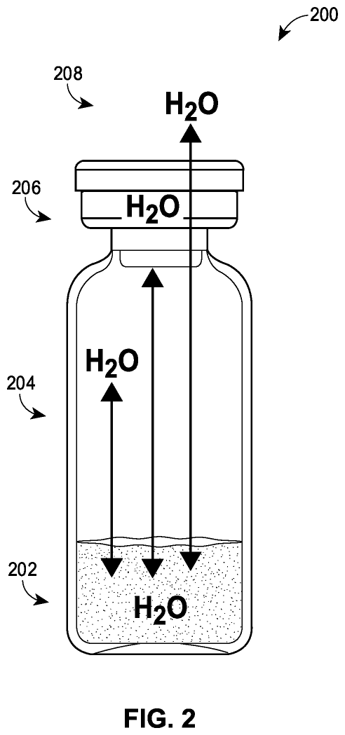

FIG. 2 depicts water transmission mechanisms that may be simulated by the computational model 146. For a virtual/simulated vial 200, the computational model 146 may account for (1) water exchange between air 204 within the vial 200 and a cake 202, (2) water exchange between a stopper 206 and the air 204 within the vial 200 (via the bottom edge of the stopper 206), and (3) water exchange between the stopper 206 and an ambient environment 208 external to the vial 200 (via the top edge of the stopper 206). The vial 200 is typically glass, with any water transfer directly between the walls of the vial 200 and the ambient environment 208 being negligible. The stopper 206 is typically rubber. Over time, water is exchanged between the ambient environment 208, the stopper 206, the internal air 204 and the cake 202. The equilibrium water moisture content for the cake 202 and the stopper 206, across a range of relative humidity values and across a range of temperatures, can be experimentally determined and represented by sorption/desorption moisture isotherms for the cake 202 and stopper 206. These isotherms, and their role in the computational model 146, are discussed in further detail below.

A more specific implementation of the computational model 146 will now be described, with reference to the example vial 200 of FIG. 2. In this example implementation, the computational model 146 is subject to a number of assumptions: (1) the vial 200 and stopper 206 are axi-symmetrically shaped around the same central axis; (2) the diffusivity of water (and possibly other physical properties) is constant for the stopper 206, the air 204, and the cake 202 over the simulated time period, which is typically a valid assumption given the limited shelf life of drug products; (3) the ideal gas law applies at all times for water vapor in the vial 200; (4) moisture transport by diffusion, in and out of the vial, is the primary physical phenomenon of interest (i.e., other phenomena, such as air leaking through the stopper/vial contact area, heat transfer resistances, and deformation of the stopper and cake are relatively inconsequential over the shelf life of the drug product); (4) hysteresis between stopper sorption/desorption cycle isotherms is negligible/ignored; and (5) moisture diffuses through the air 204 and cake 202 with the same diffusivity coefficient, because the cake 202 is assumed to be porous and moisture therefore diffuses through the air even within the cake domain. In other implementations, the computational model 146 is subject to more, fewer, and/or different assumptions. For example, asymmetries around the central axis may be captured if the computational model 146 is a three-dimensional rather than a two-dimensional FEA model.

Initially, the computational model 146 defines the spatial environment to be simulated (e.g., the contours of the mesh for FEA modeling and the boundaries of each domain/medium) based on model inputs that specify the vial and stopper configuration. For example, a user may indicate a vial type and stopper type, and the moisture prediction application 130 may use the indicated types to retrieve precise dimensions known to correspond to those component types.

Generally, the computational model 146 of this example applies the following binary diffusion of moisture equation in each medium (cake 202, air 204, and stopper 206):

$$\\ \begin{matrix}\frac{\partial C}{\partial t} = {D}_{m , i} \frac{{\partial }^{2} C}{\partial {x}^{2}} & Equation ( 1 )\end{matrix}$$

where C is the water concentration (e.g., in kg/m³ or mol/m³), D_{m,i} is the diffusivity coefficient of moisture m in the respective medium i (e.g., in m²/s), t is time (e.g., in seconds), and x is position (e.g., in meters). The prediction unit 140 may solve Equation (1) by transforming the equation into a weak partial differential equation by finite-element analysis, and directly solving the resulting algebraic equations until the model converges to a final solution at each virtual time step. The prediction unit 140 may use any suitable tolerance (e.g., 10⁻²) when checking for model convergence.

The computational model 146 of this example applies an initial condition by setting the initial water concentration in each medium:

C=C_{0,i} Equation (2)

The computational model 146 may assume that the air 204 in the vial 200 is initially completely dry (C_{air_init}=0), while initial water concentrations for the stopper 206 and cake 202 may be based on model inputs. In particular, the computational model 146 may calculate the water concentration in the stopper 206 and product (cake 202) as:

$$\\ \begin{matrix}{C}_{stopper \_ init} = \frac{{stopper}_{init \_ weight}}{{volume}_{stopper}} & Equation ( 3 )\end{matrix}$$

$$\\ \begin{matrix}{C}_{product \_ init} = \frac{{product}_{init \_ weight}}{100 \times {ρ}_{product}} & Equation ( 4 )\end{matrix}$$

where stopper_{init_weight} is the starting weight of the stopper 206, product_{init_weight} is the starting weight of the product in the cake 202, and ρ_{product} is the density of the product in the cake 202 (e.g., in kg/m³). The parameters stopper_{init_weight}, product_{init_weight}, and ρ_{product} may be model inputs entered by a user, or values that the moisture prediction application 130 calculates based on inputs entered by a user (e.g., based on a stopper type, drug product type, and/or SKU identifier entered by the user, etc.).

The computational model 146 of this example also applies boundary conditions at four interfaces. Between the ambient environment 208 and the top of the stopper 206, the computational model 146 applies the boundary condition:

$$\\ \begin{matrix}{C}_{stopper \_ top} = {ρ}_{stopper} \frac{{isotherm}_{stopper} ( RH )}{100} & Equation ( 5 )\end{matrix}$$

where ρₛₜₒₚₚₑᵣ is the density of the stopper 206 (e.g., in kg/m³), and isothermₛₜₒₚₚₑᵣ(RH) is the sorption/desorption isotherm value for the stopper 206 as a function of relative humidity (RH). For a given ambient temperature, relative humidity may be defined as the ratio of the observed vapor pressure to the saturation vapor pressure at the same conditions. Alternatively, relative humidity may be defined as the ratio of the mole fraction of water vapor in the atmosphere to the mole fraction of water vapor in the atmosphere at saturation. For a given set of storage conditions, relative humidity may be experimentally measured by measuring the partial pressure of water in the air. Relative humidity may be expressed as a percentage or fraction.

For the interface between the bottom of the stopper 206 and the air 204 within the vial 200, the computational model 146 calculates a partition coefficient:

$$\\ \begin{matrix}{K}_{stopper} = \frac{{C}_{stopper}}{{C}_{air}} & Equation ( 6 )\end{matrix}$$

$$\\ where$$

$$\\ \begin{matrix}{C}_{stopper} = \frac{{ρ}_{stopper} \times {isotherm}_{stopper} ( RH )}{100} & Equation ( 7 )\end{matrix}$$

For the interface between the product/cake 202 and the air 204 within the vial 200, the computational model 146 calculates another partition coefficient:

$$\\ \begin{matrix}{K}_{product} = \frac{{C}_{product}}{{C}_{air}} & Equation ( 8 )\end{matrix}$$

$$\\ where$$

$$\\ \begin{matrix}{C}_{product} = \frac{{ρ}_{product} \times {isotherm}_{product} ( RH )}{100} & Equation ( 9 )\end{matrix}$$

and where isotherm_{product}(RH) is the sorption/desorption isotherm value for the product/cake 202 as a function of relative humidity (RH). The computational model 146 of this example requires that the partition coefficients Kₛₜₒₚₚₑᵣ and K_{product} hold true at the stopper-air and product-air interfaces, respectively, at equilibrium.

The computational model 146 sets the water diffusive fluxes to be equal on both sides of each interface (i.e., the air-stopper interface and the air-cake interface):

$$\\ \begin{matrix}{( {D}_{m , i} \frac{\partial C}{\partial x} )}^{+} = {( {D}_{m , i} \frac{\partial C}{\partial x} )}^{-} & Equation ( 10 )\end{matrix}$$

At the bottom of the cake 202, the computational model 146 sets the water diffusion to be zero:

$$\\ \begin{matrix}\frac{\partial C}{\partial x} = 0 & Equation ( 11 )\end{matrix}$$

A number of the model parameters in Equations (1) through (11) may be experimentally measured. For example, the diffusion coefficient in the stopper 206 may be measured for the type of stopper being simulated. The air diffusion coefficient may be used for moisture diffusion through not only the air 204, but also the cake 202, due to the typically porous nature of the cake 202. The initial moisture content for the cake 202 and stopper 206 may also be experimentally measured for a given drug product and stopper combination.

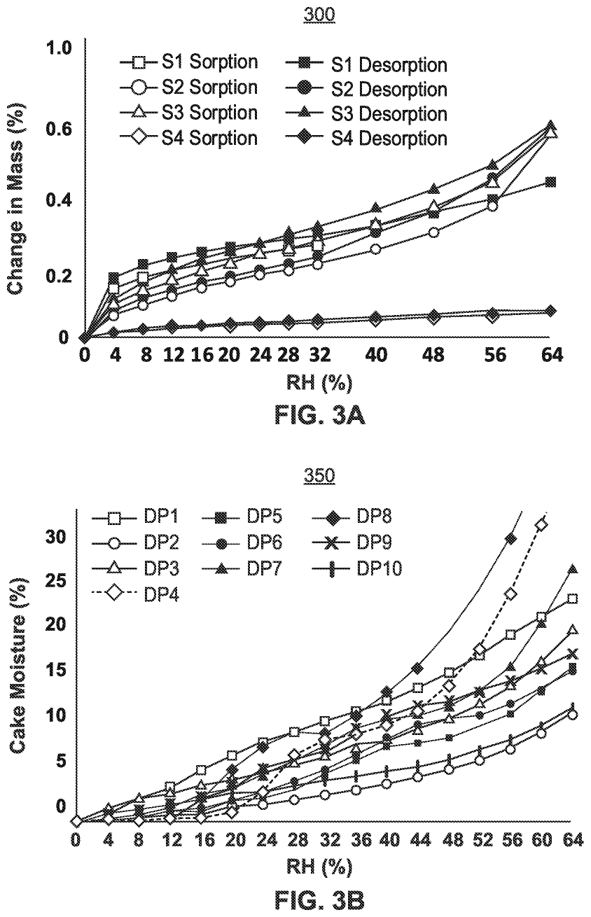

The moisture isotherms for the stopper 206 (for Equations (5) and (7)) and product/cake 202 (for Equation (9)) may also be experimentally measured. For a particular stopper type, for example, a sample stopper may be cut into smaller portions (e.g., cryomilled) and then tested across different temperatures and relative humidity levels. For a particular drug product, a core/sample of the lyophilized product (cake) may be tested across different temperatures and relative humidity levels. FIG. 3A depicts example sorption and desorption isotherms 300 for four different types of stoppers, labeled S1 through S4, and FIG. 3B depicts example moisture isotherms 350 for ten different types of drug product, labeled DP1 through DP10. In each case, the prediction unit 140 can determine the change in mass (i.e., in the stopper 206 or cake 202) percentage-wise relative to the dry state, using relative humidity (RH) as the argument of the function.

In some implementations, after the isotherms (e.g., isotherms 300 and 350) are determined experimentally at a number of different temperatures, the isotherms are incorporated into the computational model 146 as piecewise cubic interpolation functions with constant extrapolations. For example, if isotherms for the stopper 206 (and/or product/cake 202) are measured at 5, 25, and 40 degrees Celsius, the computational model 146 may employ a generally smooth function that allows for any input temperature by multiplying each discrete isotherm function by a basis function, as follows:

$$\\ \begin{matrix}Isotherm Value ( change in mass ) = {isotherm}_{05} ( RH ) , & Equation ( 12 )\end{matrix}$$

$$\\ for T \leq 5 = ( {isotherm}_{05} ( RH ) ( 1 - \frac{T - 5}{20} ) + {isotherm}_{25} ( RH ) ( \frac{T - 5}{20} ) ) ,$$

$$\\ for 5 < T \leq 25 = ( {isotherm}_{25} ( RH ) ( 1 - \frac{T - 25}{15} ) + {isotherm}_{40} ( RH ) ( \frac{T - 25}{15} ) ) ,$$

$$\\ for 25 < T \leq 40 = {isotherm}_{40} ( RH ) , for T > 40$$

where isotherm₀₅(RH) is the isotherm measured at 5 degrees Celsius, isotherm₂₅(RH) is the isotherm measured at 25 degrees Celsius, isotherm₄₀(RH) is the isotherm measured at 40 degrees Celsius, and T is the temperature in Celsius. In some implementations, the isotherms may be averages of sorption and desorption isotherms, on the assumption that the difference between the two has low significance. In other implementations, the computational model 146 uses different isotherms for sorption and desorption cycles in the stopper 206 and/or product/cake 202.

In other implementations, the computational model 146 may implement equations other than those shown above. For example, the computational model 146 may utilize more, fewer, and/or different model input parameters, account for more, fewer, and/or different water transmission mechanisms, perform calculations in terms of water amounts (e.g., water mass) rather than water concentration, and so on.

FIG. 4A depicts an example user interface 400 that may be presented to a user of the system 100 of FIG. 1. The user interface 400 may be generated by the GUI unit 142, for example, and may be presented by the display device 124 and/or a similar display device of another computing system.

The user interface 400 enables a user to enter a number of model parameters, and/or information from which the moisture prediction application 130 (or another application of computing system 106 or a different computing system) can calculate or derive one or more model parameters. In the example shown, a user can enter, or select (e.g., from a drop-down menu), a drug product type (here, “DP1”), a cake density, a fill weight, an initial product weight (%), a vial type (here, “V5”), a stopper type (here, “S3”), an initial stopper weight, and storage conditions. In this particular example, the computational model 146 can account for different ambient environment 208 conditions (relative humidity and temperature) at different virtual time periods (e.g., corresponding to long-term storage, shipping, and then short-term storage). In other implementations, the computational model 146 assumes constant ambient conditions.

The moisture prediction application 130 may use the user-selected drug product type, vial type, and stopper type to obtain a number of model inputs. For example, moisture prediction application 130 may use the selected vial and stopper types to retrieve geometries for the vial 200 and stopper 206, use the selected stopper type to determine which experimental stopper moisture isotherms (or which function/approximation of such isotherms, etc.) to apply in Equations (5) and (7), use the selected drug product type to determine which experimental drug product moisture isotherms (or which function/approximation of such isotherms, etc.) to apply in Equation (9), and use the selected drug product and stopper types to determine which experimental diffusivity coefficients to use for the drug product and stopper, respectively, in Equation (1). The moisture prediction application 130 may also use the entered initial stopper weight for Equation (3), the entered initial product weight for Equation (4), and the entered cake density for Equations (4) and (9). Moreover, the moisture prediction application 130 may use the temperature and relative humidity storage values for the indicated virtual time periods (e.g., to determine the appropriate stopper isotherm values in Equation (12), as applied in Equation (5)).

GUI unit 142 may also provide other user controls for setting model parameters. As just one example, the user interface 400 may include a field or other control that enables a user to set the length of each virtual time step. A user may select or enter inputs on the user interface 400 using the user input device 126.

FIG. 4B depicts an example output 420 that may be presented to a user of the system 100 of FIG. 1. The output 420 may be presented on a user interface generated by the GUI unit 142, for example, and may be displayed by the display device 124 and/or a similar display device of another computing system. As seen in FIG. 4B, the output 420 is a plot of cake moisture (as a percentage) over the simulated/virtual time steps.

In some implementations, the GUI unit 142 may instead, or also, generate other outputs. For example, the GUI unit 142 may output a final water concentration or amount in the cake (i.e., at a final virtual time step). In some implementations, the GUI unit 142 also provides a graphic illustration of the distribution or contours of water concentration throughout the cake, the vial interior (air), and the stopper. The GUI unit 142 may provide a fixed “snapshot” of the water concentration distribution at a final virtual time step or may provide a dynamic indicator of the water concentration distribution over time (e.g., in response to the user sliding a virtual button along a time scale, or automatically in a “playback” mode, etc.). FIG. 4B provides one example output 440 in which the distribution of water concentration is shown over time, at three stages, with time increasing from left to right. The GUI unit 142 may use any suitable visual indicator to indicate different water concentrations in different spatial areas (e.g., at a resolution level commensurate with the mesh element size for the finite-element analysis). In some implementations, for example, a first type of visual indicator (e.g., darkness/shading) is used to distinguish the areas associated with each domain/medium (cake, air, and stopper), while a different, second type of visual indicator (e.g., color) is used to represent the range of water concentrations within each domain/medium, or vice versa. The GUI unit 142 may also provide other information helpful to the user (e.g., stopper moisture over time, an indication of which portions of the plot of output 420 correspond to which user-entered storage conditions, etc.).

The information provided by GUI unit 142 (based on the output(s) of computational model 146) can be used in any of various ways, depending on the implementation and/or scenario. For example, a user (e.g., engineering team) can conduct multiple simulations using different stopper and/or vial types to determine which stopper/vial combination provides the best shelf life stability (or provides the best trade-off between cost and shelf life stability, etc.) for a particular drug product. As another example, a user can determine optimal or otherwise desired storage conditions (possibly including shipping conditions, etc.) for a particular drug product, vial, and stopper combination by varying the temperature and relative humidity levels (e.g., via the user interface 400). As yet another example, a user can determine a stopper drying target (e.g., the optimal, or maximum permissible, starting moisture in the stopper) for a new type of stopper.

In some implementations and/or scenarios, the output information can be used to facilitate nearly instant responsiveness to out-of-spec or otherwise unexpected moisture conditions in the cake and/or stopper during or after the lyophilization process, or other process deviations. Moreover, the output information can be used to further understanding of physical mechanisms affecting stability (e.g., to augment experimental data by providing insight into water transport phenomena that cannot be measured directly in drug products, or to investigate secondary drying conditions for a lyophilization cycle, etc.), or for determining other beneficial process parameters (e.g., to determine how small the cake and/or how thin the stopper can be made, to determine environmental controls required for Karl Fischer sample preparation, etc.), and so on.

The techniques disclosed herein were validated by running a number of trials. For 54 trials with model input parameters of “stopper type,” “vial type,” “drug product,” “starting stopper moisture,” “fill weight”, “ambient temperature,” and “ambient humidity,” 90% of the actual measured water concentrations fell within the predicted cake water concentration bounds. The lower and upper bounds for the cake water concentration were obtained by scaling the stopper moisture isotherm by [0.82, 1.18].

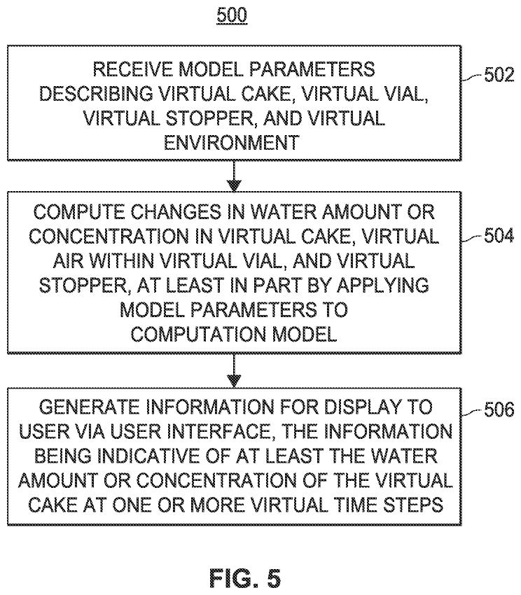

FIG. 5 is a flow diagram of an example method 500 of predicting stability of a lyophilized drug product stored in a vial. The method 500 may be implemented by processing hardware within a system such as the system 100 of FIG. 1 (e.g., in whole or in part by the processing unit 120, when executing the instructions of the moisture prediction application 130).

At block 502 of the method 500, model parameters are received. The model parameters describe a virtual cake (lyophilized drug product), a virtual vial, a virtual stopper, and a virtual ambient environment. In some example implementations, the model parameters include geometries of the virtual vial and virtual stopper, other stopper parameters (e.g., moisture diffusivity, density, starting water amount or concentration, etc.), drug product/cake parameters (e.g., starting weight and/or density, starting water amount or concentration, etc.), ambient environment parameters (e.g., temperature, relative humidity, etc.), and/or other relevant parameters. In one implementation, the model parameters include some or all of the input parameters used in Equations (1) through (12) above.

Block 502 may include receiving model parameters directly from a user via a user entry or selection (e.g., via a GUI such as user interface 400), retrieving model parameters based on such user-provided information (e.g., based on an entered stopper, vial, and/or drug product type, etc.), and/or receiving model parameters in a file format, for example. In some implementations, one or more of the model parameters are not provided as run-specific inputs, but rather are retrieved at block 502 when loading the computational model. For example, the model developer may have programmed a set of functions representing stopper-specific and drug product-specific moisture isotherms, and block 502 may include identifying and/or retrieving the appropriate function(s) based on user inputs (e.g., stopper type and drug product type). As another example, the model developer may have obtained sets of stopper and/or vial blueprints representing axisymmetric cross-sectional geometries (e.g., using a tool such as WebPlotDigitalizer®), and block 502 may include identifying and/or retrieving the appropriate blueprint data based on user inputs (e.g., stopper and/or vial type). Block 502 may include retrieving some or all of the model parameters based on drug product SKU data (e.g., retrieving fill weight, initial product moisture content, and/or initial stopper weight associated with a SKU, and/or determining geometries associates with a vial and stopper type associated with a SKU, etc.).

At block 504, a change in water amount or concentration is computed in each of (1) the virtual cake, (2) virtual air within the virtual vial, and (3) the virtual stopper, at each of a plurality of virtual time steps (e.g., every day, or every 10 days, etc.), by implementing a computational model (e.g., computational model 146). Block 504 includes applying the model parameters received at block 502 to the computational model. The model may be a finite-element analysis model, such that block 504 includes computing the changes in water amount or concentration by (at each of at least some of the virtual time steps) computing a water amount or concentration and a water mass flux for each of multiple, discrete spatial elements within the virtual cake, air, and stopper. The model may be a two-dimensional, axisymmetric model that assumes symmetric vial and stopper configurations (around the central axis of the vial/stopper). Alternatively, at the cost of increased processing time and/or power, the model may account for three-dimensional asymmetries in the stopper and/or vial.

In some implementations, block 504 includes applying boundary conditions at a number of simulated interfaces. For example, block 504 may include applying at least a first boundary condition between an external surface of the virtual stopper and the virtual ambient environment, a second boundary condition between an internal surface of the virtual stopper and the virtual air within the vial, and a third boundary condition between the virtual air within the vial and the virtual cake. Applying at least some of these boundary conditions may include computing partition coefficients (e.g., a first partition coefficient determined using a function representation of a stopper-air sorption and/or desorption moisture isotherm, and a second partition coefficient determined using a function representation of a cake-air sorption and/or desorption moisture isotherm), such as in the example of Equations (6) and (8) above.

Block 504 may occur in response to a user activating an interactive control, such as a virtual “run” button (e.g., on a GUI similar to user interface 400), for example.

At block 506, information is generated for display to a user via a user interface. The information is indicative of, at least, the water amount or concentration of the virtual cake at one or more of the virtual time steps (e.g., the output 420 and/or 440). For example, the generated information may indicate the water amount or concentration of the virtual cake (as determined by the model) as a function of time, and/or a final water amount or concentration of the virtual cake (i.e., at the last virtual time step). Alternatively or additionally, the information may indicate a change in weight of the virtual cake over a virtual time period. As yet another example, the information may indicate whether the water amount or concentration of the virtual cake at a particular time step (or multiple steps) of the virtual time steps satisfies one or more stability criteria associated with the lyophilized drug product (e.g., a maximum moisture before cake collapse is likely). In some implementations, block 506 includes controlling/causing a display device (e.g., display device 124) to present the information on a GUI.

Block 506 may occur in response to a user activating an interactive control, such as a virtual “report” button (e.g., on a GUI similar to user interface 400), or may occur automatically after the model is executed, for example.

In some implementations, the method 500 includes one or more additional blocks not shown in FIG. 5. For example, the method 500 may include one or more additional blocks in which the displayed information generated at block 506 is used (e.g., by a human user or team, or automatically by processing hardware) to select a stopper and/or vial type to use for a real-world lyophilized drug product, to select an ambient environment temperature and/or humidity (e.g., relative humidity) to maintain for the lyophilized drug product, to select a stopper drying target (e.g., starting stopper moisture), and/or for other purposes. Other actions are also possible, such as rejecting a drug product batch or sample based on model outputs, displayed at block 506, that indicate the cake moisture will likely exceed a permissible threshold before the expiration of a shelf life normally associated with that drug product.

Additional considerations pertaining to this disclosure will now be addressed.

Some of the figures described herein illustrate example block diagrams having one or more functional components. It will be understood that such block diagrams are for illustrative purposes and the devices described and shown may have additional, fewer, or alternate components than those illustrated. Additionally, in various embodiments, the components (as well as the functionality provided by the respective components) may be associated with or otherwise integrated as part of any suitable components.

Embodiments of the disclosure relate to a non-transitory computer-readable storage medium having computer code thereon for performing various computer-implemented operations. The term “computer-readable storage medium” is used herein to include any medium that is capable of storing or encoding a sequence of instructions or computer codes for performing the operations, methodologies, and techniques described herein. The media and computer code may be those specially designed and constructed for the purposes of the embodiments of the disclosure, or they may be of the kind well known and available to those having skill in the computer software arts. Examples of computer-readable storage media include, but are not limited to: magnetic media such as hard disks, floppy disks, and magnetic tape; optical media such as CD-ROMs and holographic devices; magneto-optical media such as optical disks; and hardware devices that are specially configured to store and execute program code, such as ASICs, programmable logic devices (“PLDs”), and ROM and RAM devices.

Examples of computer code include machine code, such as produced by a compiler, and files containing higher-level code that are executed by a computer using an interpreter or a compiler. For example, an embodiment of the disclosure may be implemented using Java, C++, or other object-oriented programming language and development tools. Additional examples of computer code include encrypted code and compressed code. Moreover, an embodiment of the disclosure may be downloaded as a computer program product, which may be transferred from a remote computer (e.g., a server computer) to a requesting computer (e.g., a client computer or a different server computer) via a transmission channel. Another embodiment of the disclosure may be implemented in hardwired circuitry in place of, or in combination with, machine-executable software instructions.

As used herein, the singular terms “a,” “an,” and “the” may include plural referents, unless the context clearly dictates otherwise.

As used herein, the terms “approximately,” “substantially,” “substantial” and “about” are used to describe and account for small variations. When used in conjunction with an event or circumstance, the terms can refer to instances in which the event or circumstance occurs precisely as well as instances in which the event or circumstance occurs to a close approximation. For example, when used in conjunction with a numerical value, the terms can refer to a range of variation less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, two numerical values can be deemed to be “substantially” the same if a difference between the values is less than or equal to ±10% of an average of the values, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%.

Additionally, amounts, ratios, and other numerical values are sometimes presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified.

While the present disclosure has been described and illustrated with reference to specific embodiments thereof, these descriptions and illustrations do not limit the present disclosure. It should be understood by those skilled in the art that various changes may be made and equivalents may be substituted without departing from the true spirit and scope of the present disclosure as defined by the appended claims. The illustrations are not necessarily drawn to scale. There may be distinctions between the artistic renditions in the present disclosure and the actual apparatus due to manufacturing processes, tolerances and/or other reasons. There may be other embodiments of the present disclosure which are not specifically illustrated. The specification (other than the claims) and drawings are to be regarded as illustrative rather than restrictive. Modifications may be made to adapt a particular situation, material, composition of matter, technique, or process to the objective, spirit and scope of the present disclosure. All such modifications are intended to be within the scope of the claims appended hereto. While the techniques disclosed herein have been described with reference to particular operations performed in a particular order, it will be understood that these operations may be combined, sub-divided, or re-ordered to form an equivalent technique without departing from the teachings of the present disclosure. Accordingly, unless specifically indicated herein, the order and grouping of the operations are not limitations of the present disclosure.

1. A method of computational modeling to predict stability of a lyophilized drug product, the method comprising:
receiving, by processing hardware of a computing system, model parameters describing a virtual cake, a virtual vial, a virtual stopper, and a virtual ambient environment;
computing, by the processing hardware implementing a computational model and at each of a plurality of virtual time steps, a change in water amount or concentration in each of (i) the virtual cake, (ii) virtual air within the virtual vial, and (iii) the virtual stopper, wherein computing the changes in water amount or concentration includes applying the model parameters to the computational model; and
generating, by the processing hardware, information for display to a user via a user interface, the information being indicative of at least the water amount or concentration of the virtual cake at one or more time steps of the plurality of virtual time steps.

2. The method of claim 1, wherein computing the changes in water amount or concentration further includes:
applying boundary conditions (i) between an external surface of the virtual stopper and the virtual ambient environment, (ii) between an internal surface of the virtual stopper and the virtual air, and (iii) between the virtual air and the virtual cake

3. The method of claim 2, wherein applying the boundary conditions includes computing partition coefficients using function representations of one or both of:
a stopper-air sorption and/or desorption moisture isotherm; and
a cake-air sorption and/or desorption moisture isotherm.

4. The method of claim 1, wherein the computational model is a finite-element analysis (FEA) model, such that computing the changes in water amount or concentration includes computing, at each of at least some of the plurality of virtual time steps, a water amount or concentration and a water mass flux for each of a plurality of discrete spatial elements within the virtual cake, the virtual air, and the virtual stopper.

5. The method of claim 4, wherein the FEA model is a two-dimensional axisymmetric model that assumes symmetric vial and stopper configurations.

6. The method of claim 1, wherein the model parameters include geometries of the virtual vial and the virtual stopper.

7. The method of claim 1, wherein the model parameters include one or more of:
a moisture diffusivity coefficient of the virtual stopper;
a density of the virtual stopper; or
a starting water amount or concentration of the virtual stopper.

8. The method of claim 1, wherein the model parameters include one or both of:
a starting weight and/or density of the virtual cake; and
a starting water amount or concentration of the virtual cake.

9. The method of claim 1, wherein the model parameters include one or both of:
a temperature of the virtual ambient environment; and
a humidity of the virtual ambient environment.

10. The method of claim 1, wherein the information is indicative of the water amount or concentration of the virtual cake as a function of time.

11. The method of claim 1, wherein the information is indicative of a change in weight of the virtual cake.

12. The method of claim 1, wherein the information is indicative of whether the water amount or concentration of the virtual cake at a particular time step, or steps, of the plurality of virtual time steps satisfies one or more stability criteria associated with the lyophilized drug product.

13. The method of claim 1, further comprising using the displayed information to select one or more of:
a stopper type to use for the lyophilized drug product;
a vial type to use for the lyophilized drug product;
an ambient environment temperature to maintain for the lyophilized drug product;
an ambient environment humidity to maintain for the lyophilized drug product; or a stopper drying target.

14. A computing system comprising:
processing hardware; and
one or more memories storing instructions that, when executed by the processing hardware, cause the computing system to
receive model parameters describing a virtual cake, a virtual vial, a virtual stopper, and a virtual ambient environment,
compute, by implementing a computational model and at each of a plurality of virtual time steps, a change in water amount or concentration in each of (i) the virtual cake, (ii) virtual air within the virtual vial, and (iii) the virtual stopper, wherein computing the changes in water amount or concentration includes applying the model parameters to the computational model, and
generate information for display to a user via a user interface, the information being indicative of at least the water amount or concentration of the virtual cake at one or more time steps of the plurality of virtual time steps.

15. The computing system of claim 14, wherein computing the changes in water amount or concentration further includes:
applying boundary conditions (i) between an external surface of the virtual stopper and the virtual ambient environment, (ii) between an internal surface of the virtual stopper and the virtual air, and (iii) between the virtual air and the virtual cake

16. The computing system of claim 15, wherein applying the boundary conditions includes computing partition coefficients using function representations of one or both of:
a stopper-air sorption and/or desorption moisture isotherm; and
a cake-air sorption and/or desorption moisture isotherm.

17. The computing system of claim 14, wherein the computational model is a finite-element analysis (FEA) model, such that computing the changes in water amount or concentration includes computing, at each of at least some of the plurality of virtual time steps, a water amount or concentration and a water mass flux for each of a plurality of discrete spatial elements within the virtual cake, the virtual air, and the virtual stopper.

18. The computing system of claim 17, wherein the FEA model is a two-dimensional axisymmetric model that assumes symmetric vial and stopper configurations.

19. The computing system of claim 14, wherein the model parameters include geometries of the virtual vial and the virtual stopper.

20. The computing system of claim 14, wherein the model parameters include one or more of:
a moisture diffusivity coefficient of the virtual stopper;
a density of the virtual stopper; or
a starting water amount or concentration of the virtual stopper.

21. The computing system of claim 14, wherein the model parameters include one or both of:
a starting weight and/or density of the virtual cake; and
a starting water amount or concentration of the virtual cake.

22. The computing system of claim 14, wherein the model parameters include one or both of:
a temperature of the virtual ambient environment; and
a humidity of the virtual ambient environment.

23. The computing system of claim 14, wherein the information is indicative of the water amount or concentration of the virtual cake as a function of time.

24. The computing system of claim 14, wherein the information is indicative of a change in weight of the virtual cake.

25. The computing system of claim 14, wherein the information is indicative of whether the water amount or concentration of the virtual cake at a particular time step, or steps, of the plurality of virtual time steps satisfies one or more stability criteria associated with the lyophilized drug product.
